(19) United States
(12) Patent Application Publication
(10) Pub. No.: US 2019/0216690 A1
(43) Pub. Date: Jul. 18, 2019

(54) COATING COMPOSITIONS HAVING PARTICLES WITH DIFFERING REFRACTIVE INDICES FOR USE IN INTRAORAL SCANNING METHODS

(71) Applicant: 3M INNOVATIVE PROPERTIES COMPANY, St. Paul, MN (US)

(72) Inventors: Yizhong WANG, Woodbury, MN (US); Zhisheng YUN, Woodbury, MN (US); Bradley D. CRAIG, Lake Elmo, MN (US); Tao GONG, Woodbury, MN (US); Joel D. OXMAN, Minneapolis, MN (US)

(73) Assignee: 3M Innovative Properties Company, St. Paul, MN (US)

(21) Appl. No.: 16/302,152

(22) PCT Filed: May 17, 2017
(86) PCT No.: PCT/US2017/033132

§ 371 Date: Nov. 16, 2018

Related U.S. Application Data
(60) Provisional application No. 62/342,751, filed on May 27, 2016.

(51) Int. Cl. A61K 6/10; A61K 6/00

(52) U.S. Cl. CPC A61K 6/10; A61C 9/004; A61K 6/0005

(57) ABSTRACT

Disclosed herein are aqueous dental coating compositions and methods for enhancing contrast of intraoral surfaces for 3-dimensional digital scanning. The compositions include: an aqueous solvent system; a water soluble polymer present at no greater than 5 wt. %, based on the total weight of the aqueous dental coating composition; a plurality of first particles having a first index of refraction, and a plurality of second particles having a second index of refraction different than the first index of refraction, wherein the first particles and the second particles are substantially non-absorptive in the visible range, and wherein the aqueous dental coating composition is a dispersion or a dispersion of the first particles and second particles in the aqueous solvent system can be formed by shaking the composition for 30 seconds.

### BACKGROUND

Certain intraoral scanning systems rely upon a powder that is applied to the teeth before video imaging and subsequent three-dimensional (3D) digital impressions or models can be successfully generated. One of the challenges for successfully generating digital impressions using a multiple view geometry method is that a sufficient number of features with sufficient contrast must be obtained in the video images of the teeth. There is a wide range of teeth color and texture in the patient populace in conjunction with practical resolution limitations of the camera system that can require the application of a powder to homogenize a wide variety of imaging conditions.

Some of these scanning systems have used a white powder including titanium dioxide particles. The white powder has been deemed sufficient to provide the consistent scattering of light from the scanning wand and texture or granularity that would lead to adequate features in the video images. However, handling of particles in aerosols, for example can be messy and cause difficulty in handling, typically utilizing expensive and specially designed delivery devices to dust dental tissues. Such titanium dioxide powders can also lack sufficient adhesion to dental structures, and thus can be easily moved around by saliva, the tongue, and/or the scanner wand.

There is a continuing need for materials and methods for enhancing contrast of teeth for intraoral scanning.

### SUMMARY

In one aspect, the present disclosure provides an aqueous dental coating composition including: an aqueous solvent system; a water soluble polymer present at no greater than 5 wt. %, based on the total weight of the aqueous dental coating composition; a plurality of first particles having a first index of refraction; and a plurality of second particles having a second index of refraction different than the first index of refraction, wherein the first particles and the second particles are substantially non-absorptive in the visible range, and wherein the aqueous dental coating composition is a dispersion or a dispersion of the first particles and second particles in the aqueous solvent system can be formed by shaking the composition for 30 seconds.

In another aspect, the present disclosure provides a method of intraoral scanning including: applying an aqueous dental coating composition as described herein to an intraoral surface or model thereof to form a coating on the surface; and scanning the coated surface to form a three dimensional representation of the intraoral surface.

The aqueous dental coating compositions can be advantageous for ease of application, along with enhanced contrast enhancement, especially when used with, for example, a multiple view geometry method (e.g., multiple camera or stereo camera oral scanners) such as the oral scanner available under the trade designation 3M TRUE DEFINITION Scanner for digital impressioning from 3M. At least in some embodiments, the compositions and methods disclosed herein can overcome at least some of the problems encountered with application of powders (e.g., messy and difficult handling, utilization of expensive and specially designed delivery devices to dust dental tissues; and lack of sufficient adhesion to dental structures).

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.”

The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain situations by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Also, as used herein in connection with a measured quantity, the term “approximately” refers to that variation in the measured quantity as would be expected by the skilled artisan making the measurement and exercising a level of care commensurate with the objective of the measurement and the precision of the measuring equipment used.

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein, the term “room temperature” refers to a temperature of 20° C. to 25° C., or more often to a temperature of 21° C.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

Disclosed herein are aqueous dental coating compositions and methods for enhancing contrast of intraoral surfaces for 3-dimensional digital scanning. As used herein, the term “intraoral surface” refers to a surface of a dental object in the oral cavity of a subject. The term “dental object”, as used herein, is intended to refer broadly to subject matter specific to dentistry. This may include intraoral structures such as dentition, and more typically human dentition, such as individual teeth, quadrants, full arches, pairs of arches which may be separate or in occlusion of various types, soft tissue, and the like, as well bones and any other supporting or surrounding structures. As used herein, the term “intraoral structures” refers to both natural structures within a mouth as described above and artificial structures such as any of the dental objects described below that might be present in the mouth. Dental objects may include “restorations,” which may be generally understood to include components that restore the structure or function of existing dentition, such as crowns, bridges, veneers, inlays, onlays, amalgams, composites, and various substructures such as copings and the like, as well as temporary restorations for use while a permanent restoration is being fabricated. Dental objects may also include a “prosthesis” that replaces dentition with removable or permanent structures, such as dentures, partial dentures, implants, retained dentures, and the like. Dental objects may also include “appliances” used to correct, align, or otherwise temporarily or permanently adjust dentition, such as removable orthodontic appliances, surgical stents, bruxism appliances, snore guards, indirect bracket placement appliances, and the like. Dental objects may also include “hardware” affixed to dentition for an extended period, such as implant fixtures, implant abutments, orthodontic brackets, and other orthodontic components. Dental objects may also include “interim components” of dental manufacture such as dental models (full and/or partial), wax-ups, investment molds, and the like, as well as trays, bases, dies, and other components employed in the fabrication of restorations, prostheses, and the like. Dental objects may also be categorized as natural dental objects such as the teeth, bone, and other intraoral structures described above or as artificial dental objects such as the restorations, prostheses, appliances, hardware, and interim components of dental manufacture as described above.

The aqueous dental coating compositions include: an aqueous solvent system; a water soluble polymer present at no greater than 5 wt. %, based on the total weight of the aqueous dental coating composition; a plurality of first particles having a first index of refraction; and a plurality of second particles having a second index of refraction different than the first index of refraction, wherein the first particles and the second particles are substantially non-absorptive in the visible range, and wherein the aqueous dental coating composition is a dispersion or a dispersion of the first particles and second particles in the aqueous solvent system can be formed by shaking the composition for 30 seconds.

The aqueous dental coating compositions include an aqueous solvent system. In some embodiments, the aqueous solvent system consists of water. In certain embodiments, the aqueous solvent system includes, for example, one or more cosolvents in addition to water. The use of cosolvents can be advantageous, for example, to enhance the drying properties of the coating after application to an intraoral surface. In certain embodiments, cosolvents can also enhance the dissolution of the water soluble polymer and/or enhance the dispersiblity of the particles.

A wide variety of water miscible cosolvents can be used. Particularly useful cosolvents include, for example, lower alkanols such as ethanol.

For exemplary embodiments in which the aqueous solvent system includes water and ethanol, the solvent system can include at least 10 wt. % ethanol, at least 20 wt. % ethanol, or at least 30 wt. % ethanol, based on the total weight of the water and ethanol. For exemplary embodiments in which the aqueous solvent system includes water and ethanol, the solvent system can include at most 55 wt. % ethanol, at most 40 wt. % ethanol, or at most 30 wt. % ethanol, based on the total weight of the water and ethanol. Higher amounts of ethanol as a cosolvent are typically avoided to minimize any burning or discomfort to the patient during application or rinsing procedures.

The aqueous dental coating compositions also include a water soluble polymer. In some embodiments, the water soluble polymer advantageously enhances the bonding of the particles to the intraoral surface after application and drying of the aqueous dental coating composition to the intraoral surface.

A wide variety of safe and effective water soluble polymers can be used in the aqueous dental coating compositions. Particularly useful water soluble polymers include, but are not limited to, modified cellulose, polyethylene glycol (PEG), polyacrylic acid, polyvinyl alcohol (PVA), polyethylene oxide-polypropylene oxide copolymers (PEO-PPO), polyethylene oxide-vinyl acetate copolymers (PEO-PVAc), polyvinyl pyrrolidinone (PVP), Eudragit acrylates, or combinations thereof.

The aqueous dental coating composition includes no greater than 5 wt. % of a water soluble polymer, based on the total weight of the aqueous dental coating composition. In certain embodiments, the aqueous dental coating composition includes at least 0.1 wt. % water soluble polymer, at least 0.5 wt. % water soluble polymer, or at least 1.5 wt. % water soluble polymer, based on the total weight of the aqueous dental coating composition. In certain embodiments, the aqueous dental coating composition includes at most 5 wt. % water soluble polymer, at most 3 wt. % water soluble polymer, or at most 2 wt. % water soluble polymer, based on the total weight of the aqueous dental coating composition.

The aqueous dental coating composition further includes a plurality of first particles having a first index of refraction; and a plurality of second particles having a second index of refraction different than the first index of refraction. The combination of particles having differing indices of refraction can be advantageous for coating intraoral surfaces having enhanced contrast for 3-dimensional digital scanning. In some embodiments, the first particles and the second particles are substantially non-absorptive in the visible range. As used herein, “substantially non-absorptive” in the visible range means that the particles absorb less than 50% of incident light having a wavelength of 400 nm to 700 nm. In some embodiments, the first particles and the second particles are tooth-colored. As used herein, “tooth-colored” refers to the color of a normal, vital, healthy tooth.

In some embodiments, the plurality of first particles and the plurality of second particles have indices of diffraction that differ by at least 0.05. In some embodiments, the plurality of first particles and the plurality of second particles have indices of diffraction that differ by at least 0.1. In some embodiments, the plurality of first particles and the plurality of second particles have indices of diffraction that differ by at least 0.2.

A wide variety of particles can be used in the aqueous dental coating compositions described herein. Useful particles include organic particles, inorganic particles, and combinations thereof. Particles can be discrete primary particles, agglomerated particles, or aggregated particles. In certain embodiments, particles can be core-shell particles.

Exemplary organic particles include polymeric particles that can be in the form of beads, spheres, rods, or any other form desired. The polymeric particles can be unfilled, or filled with other fillers such as inorganic fillers to form composite particles.

Exemplary inorganic particles include, but are not limited to, metal particles, metal oxide particles, metal hydroxide particles, metal nitride particles, metal sulfide particles, metal phosphide particles, and metal salts of organic and/or inorganic acids.

Particles can be surface modified particles or non-surface modified particles. Surface modified particles include particles that have been coated with another material or otherwise treated (e.g., by oxidation or other reactions of surface groups) to provide particles in which the surface properties have been modified.

In certain embodiments, a particle can be surface treated to provide hydrophilic properties. A wide variety of surface treatments can be used for hydrophilic properties. Exemplary hydrophilic surface treatments include, but are not limited to, alcohols and polyols, glycols (e.g., ethylene glycol), polyethylene glycols, modified polyethylene glycols, amino acids, silane modified amino acids, and water soluble polymers as described herein.

In certain embodiments, a particle can be surface treated to provide hydrophobic properties. A wide variety of surface treatments can be used for hydrophobic properties. Exemplary hydrophobic surface treatments include, but are not limited to, alkyl or aryl carboxylates (e.g., decanoate salts, dodecanoate salts, and benzoate salts), alkyl sulfates (e.g., lauryl sulfate salts), alkyl or aryl sulfonates (e.g., lauryl sulfonate salts and benzene sulfonate salts), silanes (e.g., alkyl trialkoxy silanes), and siloxanes.

Useful particles include inorganic particles, including but not limited to ZrO₂ particles and CaCO₃ particles. The particles can be surface treated particles or non-surface treated particles. Particularly useful particles include, for example, non-surface treated ZrO₂ particles having an index of refraction of 2.1, and non-surface treated CaCO₃ particles having an index of refraction of 1.6.

In some embodiments, the plurality of first particles and the plurality of second particles each have an average size of 0.01 to 10 microns. In some embodiments, the plurality of first particles and the plurality of second particles each have an average size of at least 0.01 micron, at least 0.05 micron, or at least 0.5 micron. In some embodiments, the plurality of first particles and the plurality of second particles each have an average size of at most 10 microns, at most 5 microns, or at most 3 microns.

In some embodiments, the aqueous dental coating composition includes 5 to 30 wt. % of the plurality of first particles, based on the total weight of the aqueous dental coating composition. In certain embodiments, the aqueous dental coating composition includes at least 5 wt. % of the plurality of first particles, at least 10 wt. % of the plurality of first particles, or at least 15 wt. % of the plurality of first particles, based on the total weight of the aqueous dental coating composition. In certain embodiments, the aqueous dental coating composition includes at most 30 wt. % of the plurality of first particles, at most 25 wt. % of the plurality of first particles, or at most 20 wt. % of the plurality of first particles, based on the total weight of the aqueous dental coating composition.

In some embodiments, the aqueous dental coating composition includes 5 to 30 wt. % of the plurality of second particles, based on the total weight of the aqueous dental coating composition. In certain embodiments, the aqueous dental coating composition includes at least 5 wt. % of the plurality of second particles, at least 10 wt. % of the plurality of second particles, or at least 15 wt. % of the plurality of second particles, based on the total weight of the aqueous dental coating composition. In certain embodiments, the aqueous dental coating composition includes at most 30 wt. % of the plurality of second particles, at most 25 wt. % of the plurality of second particles, or at most 20 wt. % of the plurality of second particles, based on the total weight of the aqueous dental coating composition.

The aqueous dental coating compositions described herein may optionally include various additives known in the art including, but not limited to, flavorants, fluoridating agents, buffering agents, numbing agents, remineralization agents, desensitization agents, colorants, or combinations thereof. The presence of a colorant can aid in detecting that the aqueous composition has coated all the desired intraoral surfaces. The intensity of a colorant can also aid in detecting the uniformity of the coating on the intraoral surfaces.

In some embodiments, particles can be dispersed in the aqueous dental coating composition by a variety of methods known in the art. Useful methods can include, but are not limited to, agitation, shaking, ball milling, ultrasonication of the various components, or combinations thereof, typically at room temperature for a time effective to prepare the dispersion.

The viscosity of the aqueous dental coating compositions is typically low to enable application of a thin layer of the aqueous coating composition on the intraoral surface. In some embodiments, after the dispersion has been formed, a portion or all of the fillers may settle out during storage. Particularly useful aqueous dental coating compositions include those in which the particles can be redispersed by manually shaking the composition for 30 seconds.

The present disclosure further provides a method of intraoral scanning including: applying an aqueous dental coating composition as described herein to an intraoral surface or model thereof to form a coating on the surface; and scanning the coated surface to form a three dimensional representation of the intraoral surface.

A wide variety of methods can be employed to apply the aqueous dental coating composition on the intraoral surface. Exemplary methods include brushing, rinsing, sponging, spraying, or a combination thereof. In some embodiments, the aqueous dental coating composition can be applied from the composition's container or dispenser, such as a bottle, syringe, or tube. In some embodiments, a mini dental brush, a dental brush, microfiber, foam or sponge applicator, a specially designed notched brush to fit the tooth shape for fast swabbing across the arch, or a cotton Q tip can be used to rub the surface of the intraoral surface and leave a thin layer of coating on the surface. In some other embodiments, a tray applicator, a dental tray, a sponge tray, or a dental strip filled with the aqueous dental coating composition can be used. The aqueous dental coating composition can cover the intraoral surface and leave a layer of coating on the surface. In other embodiments, the aqueous dental coating composition can be sprayed (e.g. air-brushing) with a spray device or aerosol applicator onto the intraoral surface. In other embodiments, the aqueous dental coating composition can be directly painted onto the intraoral surface with a brush tip attached to a syringe. In yet other embodiments, the aqueous dental coating composition can be applied as a rinse. Once applied to the intraoral surface, the aqueous dental coating composition can be allowed to dry, or dried by blowing air, typically within 30 seconds.

In some embodiments, the aqueous dental coating composition is capable of forming a film on an intraoral surface after application to the intraoral surface and drying.

In some embodiments, the aqueous dental coating composition is capable of forming a discontinuous film on an intraoral surface after application to the intraoral surface and drying. In some other embodiments, the aqueous dental coating composition is capable of forming the film in less than about 30 seconds after application, when dried with a stream of compressed air. In some embodiments, the dried coating has an average thickness of at most 10 microns.

The aqueous dental coating composition are particularly useful with digital scanners utilizing multiple view geometry methods (e.g., multiple cameras or stereo camera scanners). Exemplary scanning systems are described in U.S. Pat. No. 7,698,014 B2 (Dunne et al.), U.S. Pat. No. 7,813,591 B2 (Paley et al.), U.S. Pat. No. 7,840,042 B2 (Kriveshko et al.), U.S. Pat. No. 7,912,257 B2 (Paley et al.), U.S. Pat. No. 7,940,260 B2 (Kriveshko), U.S. Pat. No. 7,956,862 B2 (Zhang et al.), U.S. Pat. No. 8,454,365 B2 (Boerjes et al.), U.S. Pat. No. 8,503,763 B2 (Zhang et al.), U.S. Pat. No. 8,803,958 B2 (Zhang et al.), U.S. Pat. No. 8,830,309 B2 (Rohaly et al.), U.S. Pat. No. 8,866,883 B2 (Rohaly et al.), and U.S. Pat. No. 9,191,648 B2 (Kriveshko et al.); and U.S. Patent Application Pub. Nos. 2004/0155975 A1 (Hart et al.), 2010/0281370 A1 (Rohaly et al.), and 2011/0043613 A1 (Rohaly et al.). An exemplary oral scanner is available under the trade designation 3M TRUE DEFINITION Scanner for digital impressioning from 3M.

After scanning, in certain embodiments the applied coating can be readily removed from the intraoral surface by rinsing and/or brushing with water.

### ILLUSTRATIVE EMBODIMENTS OF THE PRESENT DISCLOSURE

Various embodiments are disclosed that can provide aqueous dental coating compositions and method of using same.

Embodiment 1A is an aqueous dental coating composition comprising: an aqueous solvent system; a water soluble polymer present at no greater than 5 wt. %, based on the total weight of the aqueous dental coating composition; a plurality of first particles having a first index of refraction; and a plurality of second particles having a second index of refraction different than the first index of refraction, wherein the first particles and the second particles are substantially non-absorptive in the visible range, and wherein the aqueous dental coating composition is a dispersion or a dispersion of the first particles and second particles in the aqueous solvent system can be formed by shaking the composition for 30 seconds.

Embodiment 2A is the aqueous dental coating composition of embodiment 1A, wherein the first particles and the second particles absorb less than 50% of incident light having a wavelength of 400 nm to 700 nm.

Embodiment 3A is the aqueous dental coating composition of embodiment 1A or 2A, wherein the first particles and the second particles are tooth-colored.

Embodiment 4A is the aqueous dental coating composition of any one of embodiments 1A to 3A, wherein the second index of refraction differs from the first index of refraction by at least 0.05.

Embodiment 5A is the aqueous dental coating composition of any one of embodiments 1A to 4A, wherein the second index of refraction differs from the first index of refraction by at least 0.1.

Embodiment 6A is the aqueous dental coating composition of any one of embodiments 1A to 5A, wherein the second index of refraction differs from the first index of refraction by at least 0.2.

Embodiment 7A is the aqueous dental coating composition of any one of embodiments 1A to 6A, wherein the aqueous solvent system comprises water, or water and ethanol.

Embodiment 8A is the aqueous dental coating composition of any one of embodiments 1A to 7A, wherein the aqueous solvent system comprises 0 to 55 wt. % ethanol, based on the total weight of water and ethanol.

Embodiment 9A is the aqueous dental coating composition of any one of embodiments 1A to 8A, wherein the solvent system includes at least 10 wt. % ethanol, based on the total weight of the water and ethanol.

Embodiment 10A is the aqueous dental coating composition of any one of embodiments 1A to 9A, wherein the solvent system includes at least 20 wt. % ethanol, based on the total weight of the water and ethanol.

Embodiment 11A is the aqueous dental coating composition of any one of embodiments 1A to 10A, wherein the solvent system includes at least 30 wt. % ethanol, based on the total weight of the water and ethanol.

Embodiment 12A is the aqueous dental coating composition of any one of embodiments 1A to 11A, wherein the solvent system includes at most 55 wt. % ethanol, based on the total weight of the water and ethanol.

Embodiment 13A is the aqueous dental coating composition of any one of embodiments 1A to 12A, wherein the solvent system includes at most 40 wt. % ethanol, based on the total weight of the water and ethanol.

Embodiment 14A is the aqueous dental coating composition of any one of embodiments 1A to 13A, wherein the solvent system includes at most 30 wt. % ethanol, based on the total weight of the water and ethanol.

Embodiment 15A is the aqueous dental coating composition of any one of embodiments 1A to 14A, wherein the aqueous dental coating composition comprises 0.1 to 5 wt. % of the water soluble polymer, based on the total weight of the aqueous dental coating composition.

Embodiment 16A is the aqueous dental coating composition of any one of embodiments 1A to 15A, wherein the aqueous dental coating composition includes at least 0.1 wt. % water soluble polymer, based on the total weight of the aqueous dental coating composition.

Embodiment 17A is the aqueous dental coating composition of any one of embodiments 1A to 16A, wherein the aqueous dental coating composition includes at least 0.5 wt. % water soluble polymer, based on the total weight of the aqueous dental coating composition.

Embodiment 18A is the aqueous dental coating composition of any one of embodiments 1A to 17A, wherein the aqueous dental coating composition includes at least 1.5 wt. % water soluble polymer, based on the total weight of the aqueous dental coating composition.

Embodiment 19A is the aqueous dental coating composition of any one of embodiments 1A to 18A, wherein the aqueous dental coating composition includes at most 5 wt. % water soluble polymer, based on the total weight of the aqueous dental coating composition.

Embodiment 20A is the aqueous dental coating composition of any one of embodiments 1A to 19A, wherein the aqueous dental coating composition includes at most 3 wt. % water soluble polymer, based on the total weight of the aqueous dental coating composition.

Embodiment 21A is the aqueous dental coating composition of any one of embodiments 1A to 20A, wherein the aqueous dental coating composition includes at most 2 wt. % water soluble polymer, based on the total weight of the aqueous dental coating composition.

Embodiment 22A is the aqueous dental coating composition of any one of embodiments 1A to 21A, wherein the water soluble polymer is selected from the group consisting of modified cellulose, polyethylene glycol (PEG), polyacrylic acid, polyvinyl alcohol (PVA), polyethylene oxide-polypropylene oxide copolymers (PEO-PPO), polyethylene oxide-vinyl acetate copolymers (PEO-PVAc), polyvinyl pyrrolidinone (PVP), and combinations thereof.

Embodiment 23A is the aqueous dental coating composition of any one of embodiments 1A to 22A, wherein the plurality of first particles have an average size of 0.01 to 10 microns.

Embodiment 24A is the aqueous dental coating composition of any one of embodiments 1A to 23A, wherein the plurality of first particles have an average size of at least 0.01 micron.

Embodiment 25A is the aqueous dental coating composition of any one of embodiments 1A to 24A, wherein the plurality of first particles have an average size of at least 0.05 micron.

Embodiment 26A is the aqueous dental coating composition of any one of embodiments 1A to 25A, wherein the plurality of first particles have an average size of at least 0.5 micron.

Embodiment 27A is the aqueous dental coating composition of any one of embodiments 1A to 26A, wherein the plurality of first particles have an average size of at most 10 microns.

Embodiment 28A is the aqueous dental coating composition of any one of embodiments 1A to 27A, wherein the plurality of first particles have an average size of at most 5 microns.

Embodiment 29A is the aqueous dental coating composition of any one of embodiments 1A to 28A, wherein the plurality of first particles have an average size of at most 3 microns.

Embodiment 30A is the aqueous dental coating composition of any one of embodiments 1A to 29A, wherein the plurality of first particles comprise inorganic particles.

Embodiment 31A is the aqueous dental coating composition of any one of embodiments 1A to 30A, wherein the plurality of first particles comprise ZrO₂ particles.

Embodiment 32A is the aqueous dental coating composition of any one of embodiments 1A to 31A, wherein the aqueous dental coating composition comprises 5 to 30 wt. % of the first particles, based on the total weight of the aqueous dental coating composition.

Embodiment 33A is the aqueous dental coating composition of any one of embodiments 1A to 32A, wherein the aqueous dental coating composition includes at least 5 wt. % of the first particles, based on the total weight of the aqueous dental coating composition.

Embodiment 34A is the aqueous dental coating composition of any one of embodiments 1A to 33A, wherein the aqueous dental coating composition includes at least 10 wt. % of the first particles, based on the total weight of the aqueous dental coating composition.

Embodiment 35A is the aqueous dental coating composition of any one of embodiments 1A to 34A, wherein the aqueous dental coating composition includes at least 15 wt. % of the first particles, based on the total weight of the aqueous dental coating composition.

Embodiment 36A is the aqueous dental coating composition of any one of embodiments 1A to 35A, wherein the aqueous dental coating composition includes at most 30 wt. % of the first particles, based on the total weight of the aqueous dental coating composition.

Embodiment 37A is the aqueous dental coating composition of any one of embodiments 1A to 36A, wherein the aqueous dental coating composition includes at most 25 wt. % of the first particles, based on the total weight of the aqueous dental coating composition.

Embodiment 38A is the aqueous dental coating composition of any one of embodiments 1A to 37A, wherein the aqueous dental coating composition includes at most 20 wt. % of the first particles, based on the total weight of the aqueous dental coating composition.

Embodiment 39A is the aqueous dental coating composition of any one of embodiments 1A to 38A, wherein the plurality of second particles have an average size of 0.01 to 10 microns.

Embodiment 40A is the aqueous dental coating composition of any one of embodiments 1A to 39A, wherein the plurality of second particles have an average size of at least 0.01 micron.

Embodiment 41A is the aqueous dental coating composition of any one of embodiments 1A to 40A, wherein the plurality of second particles have an average size of at least 0.05 micron.

Embodiment 42A is the aqueous dental coating composition of any one of embodiments 1A to 41A, wherein the plurality of second particles have an average size of at least 0.5 micron.

Embodiment 43A is the aqueous dental coating composition of any one of embodiments 1A to 42A, wherein the plurality of second particles have an average size of at most 10 microns.

Embodiment 44A is the aqueous dental coating composition of any one of embodiments 1A to 43A, wherein the plurality of second particles have an average size of at most 5 microns.

Embodiment 45A is the aqueous dental coating composition of any one of embodiments 1A to 44A, wherein the plurality of second particles have an average size of at most 3 microns.

Embodiment 46A is the aqueous dental coating composition of any one of embodiments 1A to 45A, wherein the second particles comprise inorganic particles.

Embodiment 47A is the aqueous dental coating composition of any one of embodiments 1A to 46A, wherein the second particles comprise CaCO3 particles.

Embodiment 48A is the aqueous dental coating composition of any one of embodiments 1A to 47A, wherein the aqueous dental coating composition comprises 5 to 30 wt. % of the second particles, based on the total weight of the aqueous dental coating composition.

Embodiment 49A is the aqueous dental coating composition of any one of embodiments 1A to 48A, wherein the aqueous dental coating composition includes at least 5 wt. % of the second particles, based on the total weight of the aqueous dental coating composition.

Embodiment 50A is the aqueous dental coating composition of any one of embodiments 1A to 49A, wherein the aqueous dental coating composition includes at least 10 wt. % of the second particles, based on the total weight of the aqueous dental coating composition.

Embodiment 51A is the aqueous dental coating composition of any one of embodiments 1A to 50A, wherein the aqueous dental coating composition includes at least 15 wt. % of the second particles, based on the total weight of the aqueous dental coating composition.

Embodiment 52A is the aqueous dental coating composition of any one of embodiments 1A to 51A, wherein the aqueous dental coating composition includes at most 30 wt. % of the second particles, based on the total weight of the aqueous dental coating composition.

Embodiment 53A is the aqueous dental coating composition of any one of embodiments 1A to 52A, wherein the aqueous dental coating composition includes at most 25 wt. % of the second particles, based on the total weight of the aqueous dental coating composition.

Embodiment 54A is the aqueous dental coating composition of any one of embodiments 1A to 53A, wherein the aqueous dental coating composition includes at most 20 wt. % of the second particles, based on the total weight of the aqueous dental coating composition.

Embodiment 1B is a method of intraoral scanning comprising: applying an aqueous dental coating composition according to any one of embodiments 1A to 54A to an intraoral surface or model thereof to form a coating on the surface; and scanning the coated surface to form a three dimensional representation of the intraoral surface.

Embodiment 2B is the method of embodiment 1B further comprising drying the applied aqueous dental coating composition to form the coating.

Embodiment 3B is the method of embodiment 2B, wherein the dried coating has an average thickness of at most 10 microns.

Embodiment 4B is the method of any one of embodiments 1B to 3B, wherein applying comprises brushing, rinsing, sponging, spraying, or a combination thereof.

Objects and advantages of this disclosure are further illustrated by the following non-limiting examples, but the particular materials and amounts thereof recited in these examples, as well as other conditions and details, should not be construed to unduly limit this disclosure.

### EXAMPLES

TABLE 1
List of materials

| COMPONENT | SOURCE | ADDRESS |
|---|---|---|
| Ethanol 200 proof | Columbus chemical industries | Ashville, OH |
| USP | Inc. | |
| Kollicoat IR polymer | BASF | Florham Park, NJ |
| Kollidon VA64 | BASF | Florham Park, NJ |
| Zirconium dioxide | Z Tech LLC | Bow, NH |
| Calcium carbonate, | Specialty minerals Inc | Adams, MA |
| CalEssence 70 | | |
| Calcium carbonate, | Specialty minerals Inc | Adams, MA |
| CalEssence 300 | | |
| Fruit flavor | The Foote & Jenks corporation | Camden, NJ |
| Potassium Nitrate | Sigma Aldrich | St Louis MO |

### Preparation and Characterization of Materials

### Calcium Carbonate

Calcium carbonate was from specialty minerals Inc. with trade name CalEssence 70 PCC, which has a median particle size 0.7 micron and prismatic particle shape and tapped density 0.61 grams/cc.

### Zirconium Dioxide

Zirconium dioxide is from Z-tech LLC, Bow, N.H. under the trade designation “CF-Plus-HM”, having an average particle size of 1.6 microns.

### Kollicoat IR Polymer

Kollicoat IR polymer is a polyethylene glycol (PEG) grafted with polyvinyl alcohol (PVA) polymer.

### Kollidon VA64

Kollidon VA64 is a polyvinylpyrrolidinone (PVP)-polyvinylacetate (PVAc) copolymer.

### Preparation of Polymer Solutions

Polymer solutions were prepared by charging all chemicals in a glass bottle, and then mixing by magnetic stirring for 24 hours at room temperature to form a clear solution.

TABLE 2
Polymer solution (PS) preparation (gram)

| COMPONENTS | PS-1 | PS-2 |
|---|---|---|
| DI Water | 492.5 | 242.5 |
| Kollicoat IR | 7.5 | 0 |
| polymer | | |
| Kollidon VA64 | 0 | 7.5 |
| Ethanol | 0 | 250 |

### EXAMPLES

### Preparation of Dispersions

All the components were added to glass vials, then roller mixed for 2 hours on a Wheaton culture roller mixer at maximum speed.

TABLE 3
Particle dispersions (gram)

| Components | EX-1 | EX-2 | EX-3 | EX-4 | EX-5 | EX-6 | EX-7 | EX-8 | EX-9 | CE-1 |
|---|---|---|---|---|---|---|---|---|---|---|
| PS-1 | 75 | 65 | 60 | 60 | 0 | 0 | 0 | 0 | 0 | 85 |
| PS-2 | 0 | 0 | 0 | 0 | 80 | 82.9 | 75 | 75 | 75 | 0 |
| CalEssence | 10 | 15 | 20 | 25 | 10 | 7.3 | 10 | 15 | 10 | 20 |
| 70 | | | | | | | | | | |
| CalEssence | 0 | 0 | 0 | 0 | 0 | 0 | 0 | 0 | 0 | 10 |
| 300 | | | | | | | | | | |
| Zirconium | 15 | 20 | 20 | 15 | 10 | 9.8 | 15 | 10 | 10 | 0 |
| Dioxide | | | | | | | | | | |
| Fruit flavor | 0 | 0 | 0 | 0 | 0 | 0 | 0 | 0 | 1 | 0 |
| Potassium | 0 | 0 | 0 | 0 | 0 | 0 | 0 | 0 | 4 | 0 |
| Nitrate | | | | | | | | | | |

### Scanability Testing

Dispersion samples were applied to the typodont by brushing and dried with air blowing for 10 seconds to form a coating on typodont, then scanned with 3M TRUE DEFINITION Oral scanner. Samples EX-1 to EX-9 all gave fast and easy image captures by the oral scanner. Sample CE-1 failed to give a fast and easy image capture.

All cited references, patents, or patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

1. An aqueous dental coating composition comprising:
an aqueous solvent system;
a water soluble polymer present at no greater than 5 wt. %, based on the total weight of the aqueous dental coating composition;
a plurality of first particles having a first index of refraction; and
a plurality of second particles having a second index of refraction different than the first index of refraction,
wherein the first particles and the second particles are substantially non-absorptive in the visible range, and
wherein the aqueous dental coating composition is a dispersion or a dispersion of the first particles and second particles in the aqueous solvent system can be formed by shaking the composition for 30 seconds.

2. The aqueous dental coating composition of claim 1, wherein the first particles and the second particles are tooth-colored.

3. The aqueous dental coating composition of claim 1, wherein the second index of refraction differs from the first index of refraction by at least 0.05.

4. The aqueous dental coating composition of claim 1, wherein the second index of refraction differs from the first index of refraction by at least 0.1.

5. The aqueous dental coating composition of claim 1, wherein the second index of refraction differs from the first index of refraction by at least 0.2.

6. The aqueous dental coating composition of claim 1, wherein the aqueous solvent system comprises water, or water and ethanol.

7. The aqueous dental coating composition of claim 6, wherein the aqueous solvent system comprises 0 to 55 wt. % ethanol, based on the total weight of water and ethanol.

8. The aqueous dental coating composition of claim 1, wherein the aqueous dental coating composition comprises 0.1 to 5 wt. % of the water soluble polymer, based on the total weight of the aqueous dental coating composition.

9. The aqueous dental coating composition of claim 1, wherein the water soluble polymer is selected from the group consisting of modified cellulose, polyethylene glycol (PEG), polyacrylic acid, polyvinyl alcohol (PVA), polyethylene oxide-polypropylene oxide copolymers (PEO-PPO), polyethylene oxide-vinyl acetate copolymers (PEO-PVAc), polyvinyl pyrrolidinone (PVP), and combinations thereof.

10. The aqueous dental coating composition of claim 1, wherein the plurality of first particles have an average size of 0.01 to 10 microns.

11. The aqueous dental coating composition of claim 1, wherein the plurality of first particles comprise inorganic particles.

12. The aqueous dental coating composition of claim 1, wherein the plurality of first particles comprise ZrO₂ particles.

13. The aqueous dental coating composition of claim 1, wherein the aqueous dental coating composition comprises 5 to 30 wt. % of the first particles, based on the total weight of the aqueous dental coating composition.

14. The aqueous dental coating composition of claim 1, wherein the plurality of second particles have an average size of 0.01 to 10 microns.

15. The aqueous dental coating composition of claim 1, wherein the second particles comprise inorganic particles.

16. The aqueous dental coating composition of claim 1, wherein the second particles comprise CaCO₃ particles.

17. The aqueous dental coating composition of claim 1, wherein the aqueous dental coating composition comprises 5 to 30 wt. % of the second particles, based on the total weight of the aqueous dental coating composition.

18. A method of intraoral scanning comprising:
applying an aqueous dental coating composition of claim 1, to an intraoral surface or model thereof to form a coating on the surface; and
scanning the coated surface to form a three dimensional representation of the intraoral surface.

19. The method of claim 18 further comprising drying the applied aqueous dental coating composition to form the coating.

20. The method of claim 19, wherein the dried coating has an average thickness of at most 10 microns.

21. (canceled)
